(19) United States
(12) Patent Application Publication
(10) Pub. No.: US 2016/0097028 A1
(43) Pub. Date: Apr. 7, 2016

(54) MICROFLUIDIC DEVICE FOR CELL SPHEROID CULTURE AND ANALYSIS

(71) Applicant: Academia Sinica, Taipei (TW)

(72) Inventors: Yi-Chung TUNG, Taipei (TW); Chau-Hwan LEE, New Taipei City (TW); Bishnubrata PATRA, Taipei (TW)

(21) Appl. No.: 14/506,026

(22) Filed: Oct. 3, 2014

(51) Int. Cl. C12M 1/00; G01N 33/483

(52) U.S. Cl. CPC C12M 23/58; G01N 33/4833; C12M 23/02; C12M 23/20; C12M 23/22

(57) ABSTRACT

The invention relates to a microfluidic device for culturing spheroids of human or animal body cells. The device can generate ample numbers (e.g., 5000) of uniform-sized spheroids, and the spheroids can be harvested for conventional biochemistry analysis (e.g. flow cytometry). In addition, the device can be used for observing the cultured samples using selective plane illumination microscopy (SPIM). In at least one embodiment, the microfluidic device incorporates a main body; a fluid channel extending inside the main body and having two inlets and an outlet open to the outside; and a plurality of chambers for culturing cell spheroids which are formed at the underneath of the fluid channel.

### BACKGROUND OF THE INVENTION

1. Field of the Invention

The invention relates to a microfluidic device for culturing spheroids of human or animal body cells. The device can generate ample numbers (e.g. 5000) of uniform-sized spheroids, and the spheroids can be harvested for conventional biochemistry analysis (e.g. flow cytometry). In addition, the device can be used for observing the cultured samples using the selective plane illumination microscopy (SPIM).

2. Description of the Related Art

Microfluidic devices play more and more important roles for studies using spheroid cultures because of their capability of culturing cellular spheroids for several days. Recently, multi-cellular (three dimensional) tumor spheroid culture has played an important role in cancer research compared to the conventional dish-based, two-dimensional (2D) cell cultures. A multi-cellular spheroid establishes gradients in nutrients, metabolites, catabolites, and oxygen along the spheroid radius. As a result, cellular functions and responses in tissues can be better mimicked in spheroid cultures, and thus cellular spheroids improve predictive capability of assays on drug efficacies. A better pre-clinical model can therefore be established for studies on the behavior of cells, such as endothelial cells under the influences from carcinoma cells etc.

Traditional spheroid formation methods such as hanging drops, culture of cells on non-adherent surfaces, spinner flask, or NASA rotary cell culture system usually produce various sized spheroids, which is inconvenient for many biomedical applications (Friedrich et al. 2007). For instance, spheroids with various sizes are unable to provide reliable information for drug testing due to the size dependent resistance of tumor spheroids.

Recently, various spheroid formation and cultures based on microfluidics techniques have been developed. A multilayer microfluidic device with a porous membrane has employed both the spheroid formation and in-situ culture. A microfluidic array platform containing concave microwells and flat cell culture chambers for EB formation and its culture was also developed. Formation of cell spheroid culture devices posesses some drawbacks that retard their practical use. The multilayer device with semi-transparent membranes suffers from the problem of high fidelity imaging and real time monitoring. In addition, the spheroids cannot be easily harvested from the devices due to their channel designs without additional instrumentation. The conventional analysis techniques include fluorescence staining using the antibody tagged fluorophores, but most of the microfluidic devices cannot form and culture a large number of cell spheroids with uniform size and harvest them out for further conventional analysis, such as flow cytometry or western blot.

Microfluidic devices can be applied in observation and inspection of cellular spheroids with said selective plane illumination microscopy (SPIM). SPIM is an optically sectioning microscopy technique for imaging large fluorescence samples.

Although several types of microfluidic devices have been developed for formation, culture and drug testing, they are not compatible with SPIM because of the light scattering issue. In the SPIM setup, light is introduced from a lateral direction to light up the device in which the cultured cells stored therein are to be inspected. Since the light is an exciting factor, the cells exposed thereto may easily die. Thus, the arrangement of the formed cell spheroids inside the microfluidic device is critical to avoid repeated scanning of the light. However, conventional microfluidic devices cannot provide a suitable arrangement of the cell spheroids for the use in a SPIM setup when the cell spheroids in the device are illuminated therein. Therefore, there is a need to develop a microfluidic device compatible with the inspection with the light sheet of SPIM.

### SUMMARY

The present disclosure relates to microfluidic devices for culturing and harvesting 3D cell spheroids. In particular, one embodiment could be further compatible with the test with the light sheet of SPIM

In one embodiment, the microfluidic device comprises: a main body; a fluid channel extending inside the main body and having two inlets and an outlet open to the outside; and a plurality of chambers for culturing cell spheroids which are formed at the underneath of the fluid channel, wherein the fluid channel diverges to two smaller channels which lead to each of the two inlets, respectively.

In another embodiment, the microfluidic device comprises: a main body; a fluid channel extending inside the main body and having an inlet and an outlet open to the outside; and a plurality of chambers for culturing cell spheroids which are formed at the underneath of the fluid channel, wherein the fluid channel is straight.

In another embodiment, the microfluidic device comprises: a main body; a fluid channel extending inside the main body and having an inlet and an outlet open to the outside; and a plurality of chambers for culturing cell spheroids which are formed at the underneath of the fluid channel, wherein the fluid channel has several U-turns.

In a further embodiment, the microfluidic device, which is used for not only culturing cell spheroids but also observing the cultured samples using the selective plane illumination microscopy (SPIM), comprises: a transparent and cuboid main body, a fluid channel extending inside the main body and having at least one inlet and an outlet open to the outside, and a plurality of square chambers formed at the underneath of the fluid channel, wherein each of the chambers has a flat bottom, which is parallel to the bottom of the cuboid main body; each of the chambers further has four flat side walls, which are parallel to the side walls of the main body respectively, and wherein the chambers do not overlap one another when they are observed from a light sheet introduction side of the main body.

Other aspects and embodiments of the invention are also contemplated. The foregoing summary and the following detailed description are not meant to restrict the invention to any particular embodiment but are merely meant to describe some embodiments of the invention.

### BRIEF DESCRIPTION OF THE DRAWINGS

FIG. 1 is a schematic perspective view of a microfluidic device according to the first embodiment of the present disclosure;

FIG. 2 is a side view of the microfluidic device of FIG. 1;

FIG. 3A is a schematic view showing the microfluidic device of FIG. 1 in which the cell spheroids are cultured;

FIG. 3B is a schematic view showing the microfluidic device of FIG. 1 in which the cell spheroids are harvested;

FIG. 4A is a schematic perspective view of a microfluidic device according to the second embodiment of the present disclosure;

FIG. 4B is an enlarged view of the portion “A” shown in FIG. 4A;

FIG. 5A is a schematic top view of a microfluidic device according to the third embodiment of the present disclosure;

FIG. 5B is an enlarged view of the portion “B” shown in FIG. 5A;

FIG. 6A is a schematic perspective view of a microfluidic device according to the fourth embodiment of the present disclosure;

FIG. 6B is an enlarged view of the portion “C” shown in FIG. 6B;

FIG. 7 is a schematic view of the setup of a SPIM system and the microfluidic device of FIG. 6.

### DETAILED DESCRIPTION

For the purpose of promoting an understanding of the principles of the disclosure, reference will now be made to the embodiments illustrated in the drawings and specific language will be used to describe the same. It will nevertheless be understood that no limitation of the scope of the disclosure is thereby intended, such alterations and further modifications in the illustrated device, and such further applications of the principles of the disclosure as illustrated therein are contemplated as would normally occur to one skilled in the art to which the disclosure relates.

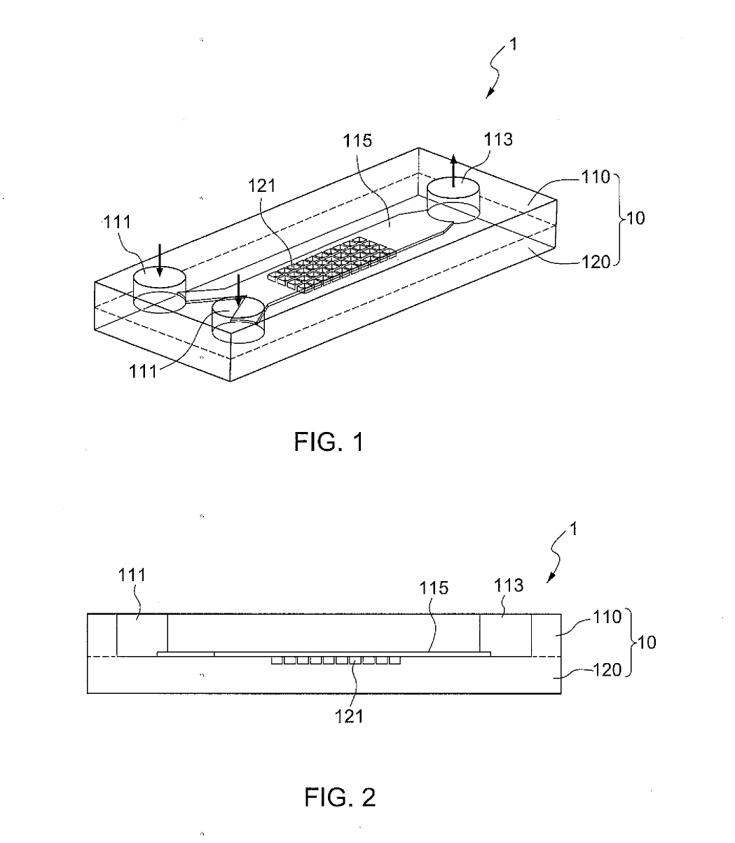

FIG. 1 illustrates a microfluidic device 1 according to a first embodiment of the present disclosure, which is used for cell spheroid formation. The microfluidic device 1 substantially comprises a main body 10, a fluid channel 115 horizontally extending inside the main body 10; the fluid channel 115 has at least one inlet and outlet, and in this embodiment, it has two inlets 111 and an outlet 113, both of which are open to the outside. A plurality of chambers 121 for culturing cell spheroids are formed underneath and open to the fluid channel 115. The fluid channel 115 diverges into two smaller channels which communicate to the outside through each of the two inlets 111, respectively. In this embodiment, the two inlets 111 and the outlet 113 are open to the top surface of the main body 10. The chambers 121 are preferably arranged in a matrix array.

As shown in FIG. 2, the main body 10 is in a cuboid shape and preferably constructed using two polydimethylsiloxane (PDMS) layers: a top layer 110 and a bottom layer 120. PDMS is broadly used to construct various microfluidic devices for cell culture because of its excellent optical transparency, manufacturability, high gas permeability and biocompatibility. The bottom layer 120 is equipped with about 5000 cubical cavities as the cell culture chambers 121 and the top layer 110 has the fluid channel 115 with at least one inlet 111 and at least one outlet 113 open to the outside. The microfluidic device 1 is fabricated by using the soft lithography replica molding process. During the process, the bottom layer 120 is aligned and irreversibly bonded with the top layer 110, wherein the fluid channel 115 passes over all the chambers 121 and the chambers 121 are open to the fluid channel 115. In one preferred embodiment, the width and length of the main body 10 is about 4×4.5 cm² with a thickness of about 1 cm; the opening of each chamber is sized at 200×200 μm² or 300×300 μm² with a depth of about 250 μm.

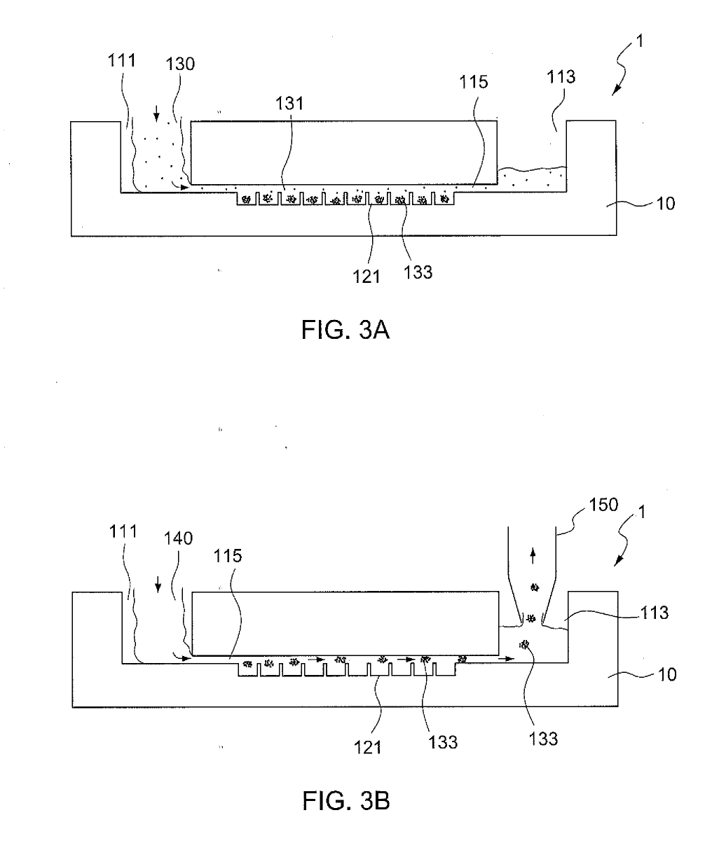

The microfluidic device 1 is used to culture three-dimensional (3D) spheroids formed from various types of cells. As shown in FIG. 3A, a cell suspension 130 is introduced from one or all of the inlets 111 with a slow flow rate into the fluid channel 115 of the microfluidic device 1. After introducing the cell suspension 130, the microfluidic device 1 is brought to a horizontal position and the fluid flows into the chambers 121 and to the outlet 113. In this way, the fluid channel 115 is full of the fluid of the cell suspension. The cells 131 are trapped and gradually deposit in the chambers 121 due to gravity and then form the cell spheroids 133 in each of the chambers 121. The microfluidic device 1 can be scaled up to form and culture more than 5000 uniformly sized cell spheroids 133 according to a user's actual need. Thus, the microfluidic device disclosed in the present invention can culture and collect a number of cell spheroids up to 100 times more than those formed in conventional microfluidic devices. Moreover, due to the capability of scaling up, the microfluidic device 1 provides a promising technique to further study cellular behaviours, including cell proliferation, migration and apoptosis in 3D spheroids under a precise mechanical, chemical and gaseous microenvironments with aids of conventional biochemical analysis methods.

After the cell spheroids 133 in the chambers 121 grow to a suitable size, they can be harvested. As shown in FIG. 3B, the fluid channel 115 is introduced to a culture medium 140 from the inlets 111 to flush the cell spheroids 133 out from the chambers 121. The culture medium 140 flushes at a sufficiently high flow rate in the fluid channel 115 so that a low-pressure area is formed over the chambers 121; the cell spheroids 133 are thus sucked from the chambers 121 and flow into the fluid channel 115. At the outlet 113, a pipette 150 is used to collect these cell spheroids 301. The cell spheroids 301 are then pipetted out from the outlet 113. In this way, the cell spheroids 133 can be harvested from the microfluidic device 1 in an efficient manner by controlling the flow rate through the fluid channel 115 with greater integrity, minus additional instrumentation and tedious procedures.

A large number of the uniformly-sized 3D cell spheroids 133 can be cultured by the microfluidic device 1 and harvested from the microfluidic device 1. Especially, the formation of different sized and/or numbers of the cell spheroids 133 can be achieved by changing the size and number of the chambers 121 of the microfluidic device 1.

Therefore, the 3D cell spheroids 133 harvested from the microfluidic device 1 are particularly suitable to be exploited for flow cytometry assays due to the ample cell numbers. This is because the conventional devices cannot culture sufficient cell spheroids, or, although some of the conventional devices such as NASA rotating vessel can culture sufficient cell spheroids, the cell spheroids are not uniformly sized.

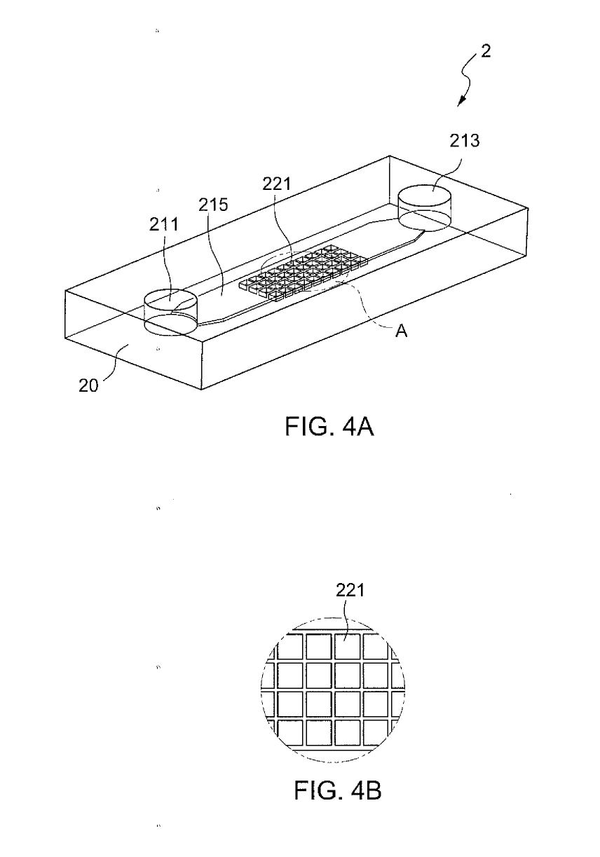

FIG. 4A illustrates another embodiment of the present invention. The microfluidic device 2 substantially comprises a main body 20, a fluid channel 215 horizontally extending inside the main body 20 and having an inlet 211 and an outlet 213 open to the outside, and a plurality of chambers 221 for culturing cell spheroids which are formed underneath and open to the fluid channel 215. The main body 10 is in a cuboid shape and is made of PDMS. The path of the fluid channel 215 is straight. The chambers 221 are preferably arranged in a matrix array (see FIG. 4B).

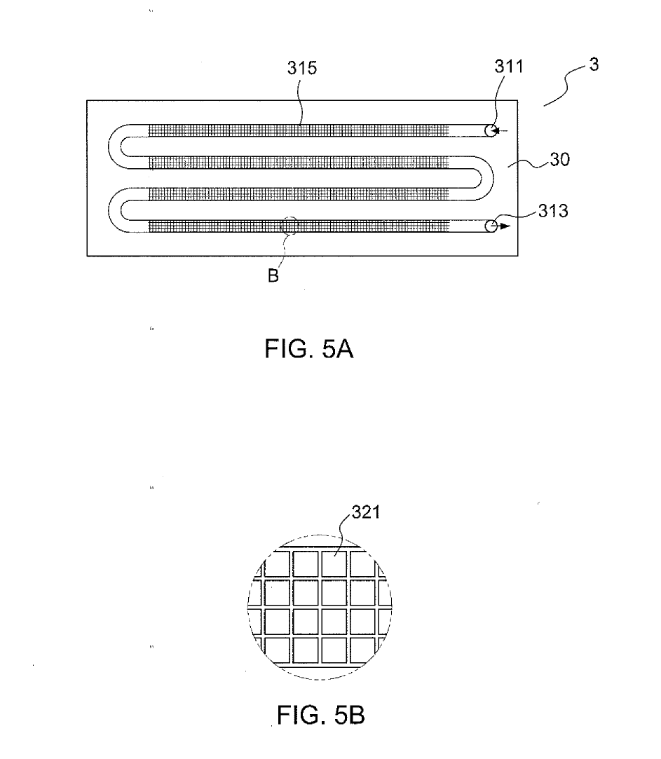

FIG. 5A illustrates a microfluidic device 3 according to a third embodiment of the present disclosure. The microfluidic device 3 has a main body 30; a fluid channel 315 horizontally extends inside the main body 30 and has an inlet 311 and an outlet 313 both open to the outside; a plurality of chambers 321 for culturing cell spheroids are formed underneath and open to the fluid channel 315. The main body 10 is in a cuboid shape and is made of PDMS. The path of the fluid channel 315 is formed as one or several U-turns. In addition, the chambers 321 underneath the fluid channel 315 are preferrably arranged in one or several matrix arrays (see FIG. 5B). The U-turn arrangement of the path of the fluid channel 315 provides a rather large space underneath the channel 315 for forming chambers 321 for culturing cell spheroids; the flow rate of the culture medium 140 for flushing the cultured cell spheroids can be maintained relatively high because of the relatively small cross section of the flow path through the fluid channel 315.

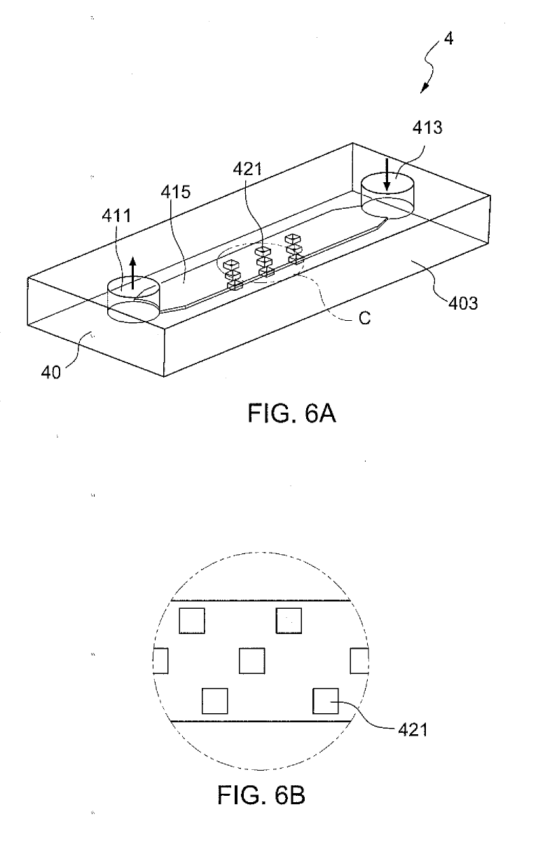

FIG. 6A illustrates a microfluidic device 4 according to a fourth embodiment of the present disclosure, which is used for not only culturing cell spheroids but also for observing the cultured samples using the selective plane illumination microscopy (SPIM). SPIM is an optically sectioning microscopy technique for imaging large fluorescence samples. In SPIM, the sample is illuminated with a sheet of light that propagates perpendicularly to the direction of observation. Therefore, a fluorescence image of a finite depth, called a sectioned image, can be formed without lateral scanning. A stack of sectioned images acquired while the sample is moved along the direction of observation can be used to form a three dimensional (3D) view of a sample, such as a cellular spheroid. The spatial resolution of SPIM can be further improved by using proper image deconvolution methods, such that a single cell can be identified in a sample of a diameter larger than 100 μm. With these unique features, SPIM is especially suitable for observing cellular behaviors in spheroids in a 3D perspective.

Referring to FIG. 6A, the microfluidic device 4 is made of transparent PDMS and substantially comprises a main body 40, a fluid channel 415 horizontally extending inside the main body 40 and having an inlet 411 and an outlet 413 open to the outside, and a plurality of chambers 421 for culturing cell spheroids, which are formed underneath and open to the fluid channel 415. The inlet 411 and the outlet 413 are open to the top surface of the main body 40. The main body 40 is made as thin as possible, and the opening of each chamber 421 is 200×200 μm² or 250×250 μm² with a depth of about 250 μm.

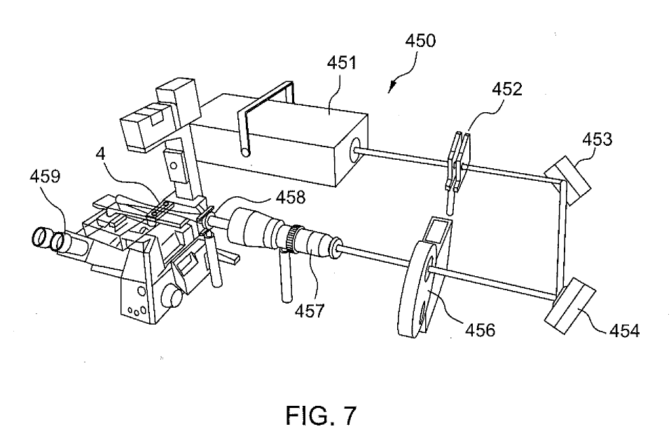

As aforementioned, after introducing the cell suspension into the main body 40 and keeping the cell suspension in the main body 40 for a period, the 3D cell spheroids are formed in the chambers 421 of the microfluidic device 4. Then, the microfluidic device 4 with the 3D cell spheroids is mounted to the SPIM system 450 (see FIG. 7) and the 3D cell spheroids formed in the chambers 421 of the microfluidic device 4 are inspected by the SPIM system 450. While inspecting the 3D cell spheroids in the chambers 421 of the microfluidic device 4 by the SPIM system 450, the light sheet of SPIM passes through the main body 40 from one side of the main body 40, which is defined as a light sheet introduction side 403, as shown in FIG. 6A. The cell spheroids in the chambers 421 are then illuminated with the light sheet and imaged in the SPIM system 450. In order to to reduce additional light scattering of excitation light sheet and emission light imaging using microscope objectives, the main body 40 is made of a cuboid shape and the light sheet introduction side 403 is preferably coated with an additional PDMS layer to ensure its optical flatness. Further, each of the chambers 421 has a flat bottom that is parallel to the bottom of the cuboid main body 40, and each of the chambers 421 further has four flat side walls that are respectively parallel to the side walls of the main body 40. Moreover, in order to minimize the light scattering and additional optical noise, the locations of the chambers 401 are arranged so that all of the chambers 401 can be uniformly illuminated with the light sheet of SPIM at a time. As shown in FIGS. 6A and 6B, the chambers 401 are arranged such that they do not overlap one another when they are observed from the light sheet introduction side 403 of the main body 40. Particularly, the chambers 421 are arranged along several parallel oblique lines not vertical to the light sheet introduction side 403 of the main body 40.

FIG. 7 shows the setup of the SPIM system 450 and the microfluidic device 4. The light source 451 is a supercontinuum laser with visible power with the wavelength of 450 to 750 nm and larger than 300 mW. The neutral density filters 452 of various transmissions are used to control the laser power. The mirrors 453 and 454 are used for reflecting the laser. The excitation filter 456 can be selected from several excitation filters mounted on a motorized filter wheel. A beam expander 457 is used to achieve the required beam diameter at the cylindrical lens 458. The cylindrical lens 458 will generate the illumination light sheet of SPIM. Further, the illumination light sheet of SPIM generated by the cylindrical lens 458 will project onto the light sheet introduction side 403 of the main body 40 of the microfluidic device 4 such that the 3D cell spheroids cultured in the chambers 421 will be illuminated with the light sheet and imaged by the CCD camera 459. In this way, the 3D cell spheroids cultured by the microfluidic device 4 can be observed by using the SPIM system 450.

The microfluidic device 4 can be applied to the SPIM system 450 to facilitate study of drugs for both pro-angiogenic and anti-angiogenic therapies. The SPIM system 450 also benefits studies on other physiological phenomena related to spheroid formation and cell-cell interactions in microenvironment established by different types of cells.

While the disclosure has been illustrated and described in detail in the drawings and foregoing description, the same is to be considered as illustrative and not restrictive in character, it being understood that only the preferred embodiments have been shown and described and that all changes and modifications that come within the spirit of the disclosure are desired to be protected.

What is claimed is:

1. A microfluidic device for culturing cell spheroids, comprising:
a main body;
a fluid channel extending inside the main body and having at least one inlet and an outlet open to the outside; and
a plurality of chambers for culturing cell spheroids, which are formed underneath and open to the fluid channel.

2. The microfluidic device according to claim 1, wherein the main body is transparent.

3. The microfluidic device according to claim 1, wherein the main body is made of PDMS.

4. The microfluidic device according to claim 1, wherein the main body is in a cuboid shape.

5. The microfluidic device according to claim 4, wherein the fluid channel extends horizontally.

6. The microfluidic device according to claim 5, wherein the inlet and the outlet are open to the top surface of the main body.

7. The microfluidic device according to claim 6, wherein the fluid channel is one of the following shapes: (i) having two inlets and diverging to two smaller channels which lead to each of the two inlets, respectively; and (ii) having at least one U-turn.

8. The microfluidic device according to claim 1, wherein the chambers are arranged in a matrix array.

9. The microfluidic device according to claim 1, wherein the chambers are substantially cubical.

10. A microfluidic device for culturing and observing cell spheroids, comprising:
a transparent and cuboid main body,
a fluid channel extending inside the main body and having at least one inlet and an outlet open to the outside, and
a plurality of square chambers formed underneath and open to the fluid channel, wherein each of the chambers has a flat bottom, which is parallel to the bottom of the cuboid main body; each of the chambers further has four flat side walls, which are respectively parallel to the side walls of the main body, and wherein the chambers do not overlap one another when they are observed from a light sheet introduction side of the main body.

11. The microfluidic device according to claim 10, wherein the chambers are arranged along several parallel oblique lines not vertical the light sheet introduction side of the main body.

12. The microfluidic device according to claim 10, wherein the fluid channel extends horizontally.

13. The microfluidic device according to claim 10, wherein the main body is made of PDMS.

14. The microfluidic device according to claim 10, wherein the light sheet introduction side is coated with a PDMS layer.

15. Equipment for inspecting cell spheroids cultured in the microfluidic device of claim 10 using selective plane illumination microscopy (SPIM).

16. A method for inspecting cell spheroids using the equipment of claim 15, comprising the following steps:
providing a fluid with cells;
injecting the fluid into the fluid channel from the inlet such that the fluid flows over the chambers;
keeping the fluid in the main body, and the cells in the fluid deposited in the chambers and gradually forming cell spheroids in each of the chambers;
emitting a light beam from the equipment;
projecting the light beam onto the light sheet introduction side of the main body to illuminate the cell spheroids; and
observing the cell spheroids by the equipment.
